(19) United States
(12) Patent Application Publication
(10) Pub. No.: US 2009/0208451 A1
(43) Pub. Date: Aug. 20, 2009

(54) HALOGEN PHOSPHONATE MONOESTERS

(75) Inventors: Vaughn Smider, Alameda, CA (US); William Heriot, Novato, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Integrigen, Inc., Novato, CA (US)

(21) Appl. No.: 10/595,422

(22) PCT Filed: Oct. 18, 2004
(86) PCT No.: PCT/US04/34386

§ 371 Date: Mar. 2, 2009

Related U.S. Application Data
(60) Provisional application No. 60/511,683, filed on Oct. 17, 2003.

(51) Int. Cl. A61K 38/21; G01N 33/53; C40B 30/04; G01N 33/543; A61K 39/385; A61K 39/44; C07K 17/02; A61P 37/06

(52) U.S. Cl. 424/85.5; 436/501; 506/9; 436/518; 424/193.1; 424/195.11; 424/178.1; 530/403

(57) ABSTRACT

The present invention relates to detection, immobilization, and production of proteolytic antibodies using halogen phosphonate monoester probes, immobilizing reagents, and antigen conjugates.

### CROSS-REFERENCES TO RELATED APPLICATIONS

This application claims the benefit of U.S. Provisional Application No. 60/511,683, filed Oct. 17, 2003 which is herein incorporated by reference in its entirety for all purposes.

### BACKGROUND OF THE INVENTION

It is known that some antibodies have catalytic ability. See Paul et al., Science, 244:1158-1162 (1989); and Li et al., J. Immunol., 154:3328-3332 (1995), discussing catalytic antibody hydrolysis of peptides or proteins. See also Shuster et al., Science, 256:665-667 (1992); and Gololobov et al., Proc. Natl. Acad. Sci. USA, 92:254-257 (1995)), discussing hydrolysis of DNA; as well as Paul, Mol. Biotechnol., 5:197-207 (1996), discussing catalytic antibodies with peroxidase activity.

Catalytic antibodies may be isolated from the natural immune repertoire, but appear to be produced at an elevated level in various autoimmune disease states (Paul, supra). Analyses of catalytic antibody components have shown that enzymatic activity often resides in the light chains, and antibody light chains isolated from multiple myeloma patients frequently demonstrate proteolytic activity (Paul, supra).

Serine proteases are a large family of proteolytic enzymes that include the digestive enzymes trypsin and chymotrypsin, components of the complement cascade and of the blood-clotting cascade, and enzymes that control the degradation and turnover of macromolecules of the extracellular matrix. The hallmark of serine proteases is the presence of a serine residue in the active catalytic site for protein cleavage. Serine proteases have a wide range of substrate specificities and diverse biological functions. Despite such diversity and often unrelated amino acid sequence, a common catalytic mechanism is shared among several sub-families of serine proteases through a very similar tertiary structure supported by a highly conserved catalytic triad of serine, histidine, and aspartate. The active site structure of one serine protease, subtilisin, is among the most studied and best understood.

Studies have provided evidence connecting proteolytic antibodies and serine proteases. Molecular modeling of the light chain of an antibody capable of hydrolyzing vasoactive intestinal polypeptide (VIP, a 28-amino acid neuropeptide) revealed an arrangement of Ser27a, His93, and Asp1 similar to the catalytic triad arrangement of a subfamily of serine proteases (Gao et al., J. Bio. Chem., 269:32389-32393 (1994)). A substitution of alanine for any one of the three amino acid residues dramatically reduced the antibody's ability to hydrolyze VIP (Gao et al., J. Bio. Chem., 253:658-664 (1995)). Diisopropyl fluorophosphate, a serine protease inhibitor, inhibits the catalytic activity of some proteolytic antibodies (Paul et al., J. Bio. Chem., 256:16128-16134, (1991)). Some proteolytic antibodies have been found to covalently bind to phosphonate diester probes, small organic molecules, and antigens (Paul, et al. J. Biol. Chem. 276: 28314-28320 (2001); Planque, et al. J. Biol. Chem. 278: 20436-20443 (2003); Paul, et al. J. Biol. Chem. 276: 28314-28320 (2001); (Rader, et al. Proc Natl Acad Sci USA 100: 5396-400 (2003); Chmura, et al. Proc Natl Acad Sci USA 98: 8480-4 (2001)). Recent work has also shown that fluorophosphonate probes could be used to profile proteins with hydrolase activity in complex proteomic mixtures (Liu, et al. Proc. Natl. Acad. Sci. 96: 14694-14699 (1999)).

The need to identify and isolate proteolytic antibodies from mixtures containing non-proteolytic antibodies is an important tool in the research and development of proteolytic antibody products. The present invention fulfills these and other needs in the art.

### BRIEF SUMMARY OF THE INVENTION

It has been discovered that, surprisingly, halogen phosphonate monoesters may be used as probes, immobilizing reagents, and antigen conjugates for use in detecting, immobilizing, and producing proteolytic antibodies. The halogen phosphonate monoester probes, immobilizing reagents, and antigen conjugates of the present invention are capable of covalently binding a proteolytic antibody.

In one aspect, the present invention provides a method for detecting the presence of a proteolytic antibody. The method includes contacting a proteolytic antibody with a halogen phosphonate monoester probe, where the halogen phosphonate monoester probe includes a detectable label. The halogen phosphonate monoester probe is allowed to covalently bind to the proteolytic antibody. Finally, after the halogen phosphonate monoester probe is covalently bound to the proteolytic antibody, the detectable label is detected.

In another aspect, the present invention provides a method for immobilizing a proteolytic antibody. The method includes contacting a proteolytic antibody with a halogen phosphonate monoester immobilizing reagent, where the halogen phosphonate monoester immobilizing reagent includes an immobilizing moiety. The halogen phosphonate monoester immobilizing reagent is allowed to covalently bind to said proteolytic antibody thereby immobilizing the proteolytic antibody.

In another aspect, the present invention provides a method of a producing proteolytic antibody in a subject. The method includes administering a halogen phosphonate monoester antigen conjugate to the subject. The subject is allowed to produce proteolytic antibodies to the halogen phosphonate monoester antigen conjugate. The proteolytic antibodies may then be isolated form the subject.

In another aspect, the present invention provides a proteolytic antibody immobilization system. The system includes a halogen phosphonate monoester immobilizing reagent and a solid support.

### BRIEF DESCRIPTION OF THE DRAWINGS

FIG. 1 shows an example of a halogen phosphonate monoester.

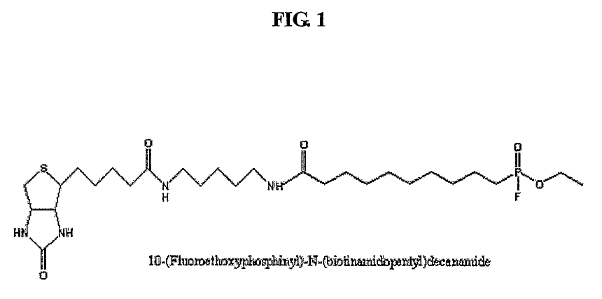

FIG. 2 shows an electrospray mass spectrum of a halogen phosphonate monoester.

FIG. 3 shows a blot containing a proteolytic V_{L} bound to a biotinylated fluorophosphonate mono ester (left), and a silver stained SDS-PAGE gel of the same reactions (right).

### DETAILED DESCRIPTION OF THE INVENTION

### Definitions

The abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The terms “arylene” and “heteroarylene” refer to the divalent derivatives of aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

Each of the above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical. Preferred substituents for each type of radical are provided below.

Substituents for the alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR″′, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radicals above, exemplary substituents for the aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally form a ring of the formula -T-C(O)—(CRR′)_{q}—U—, wherein T and U are independently NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

The compounds of the present invention may exist as salts. The present invention includes such salts. Examples of such salts include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in the art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in the art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

As used herein, an “antibody” refers to a protein functionally defined as a binding protein and structurally defined as comprising an amino acid sequence that is recognized by one of skill as being derived from the framework region of an immunoglobulin encoding gene of an animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

For preparation of monoclonal or polyclonal antibodies, any technique known in the art can be used (see, e.g., Kohler & Milstein, Nature, 256:495-497 (1975); Kozbor et al., Immunology Today, 4:72 (1983); Cole et al., Monoclonal Antibodies and Cancer Therapy, pp. 77-96. Alan R. Liss, Inc. 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies, and heteromeric F_{ab} fragments, or scFv fragments that specifically bind to selected antigens (see, e.g., McCafferty et al., supra; Marks et al., Biotechnology, 10:779-783, (1992)).

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Exemplary antibodies include single chain antibodies (antibodies that exist as a single polypeptide chain), or single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies include all those that have been displayed on phage (e.g., scFv, Fv, Fab and disulfide linked Fv (Reiter et al. (1995) Protein Eng. 8: 1323-1331). Antibodies can also include diantibodies and miniantibodies.

A “proteolytic antibody” refers to an antibody capable of catalyzing the hydrolysis of a peptide bond. Proteolytic antibodies include those antibodies with “endopeptidase activity.” Endopeptidase activity refers to the ability of an enzyme to catalyze the hydrolysis of at least one non-terminal peptide bond between two amino acid residues within a polypeptide of any length. In some embodiments, the proteolytic antibody has “specificity” for a target peptide or protein. Specificity, as used herein, refers to the ability of a proteolytic antibody to distinguish between the target protein and any other polypeptides, based on their structural difference, such that the enzymatic action upon the target protein are to a reasonable degree unique. For example, the hydrolysis of a target peptide or protein with a proteolytic antibody is deemed specific when a signal at least two times over background is detected in a proteolytic assay. Proteolytic antibodies also include those antibodies containing the catalytic active site indicative of the serine protease family. Despite the diversity in primary amino acid sequence among individual members of the family, serine protease activity is supported by a highly conserved tertiary structure, which comprises a serine-histidine-aspartate triad. Studies have shown that the aspartate residue is not always essential for catalytic activity. In an exemplary embodiment, the proteolytic antibody contains an active site “serine protease dyad.” The term “serine protease dyad,” as used herein, is the minimal structure of the catalytic site for a recombinant catalytic polypeptide to maintain at least a portion of its proteolytic activity. This structure comprises a histidine residue and a serine residue located within CDR1, CDR2, or CDR3 of an antibody V_{H} or V_{L}, where the residues are in a spatial relation to each other similar to their spatial alignment in a serine protease triad, such that the histidine can abstract the proton from the serine hydroxyl group, allowing the serine to act as a nucleophile and attack the carbonyl group of the amide bond within the protein substrate. Since the serine can bind covalently with antigens, the term “serine protease dyad” can also refer to an antibody capable of binding to a halogen phosphonate monoester probe, immobilization reagent, or antigen conjugate of the present invention.

“Antigen” refers to substances which are capable, under appropriate conditions, of inducing a specific immune response and of reacting with the products of that response, that is, with specific antibodies or specifically sensitized T-lymphocytes, or both. Antigens may be soluble substances, such as toxins and foreign proteins, particulates, such as bacteria and tissue cells, or a biomolecule, such as a peptide, polypeptide, or protein. More broadly, the term “antigen” may be used to refer to any substance to which an antibody binds, or for which antibodies are desired, regardless of whether the substance is immunogenic. For such antigens, antibodies may be identified by recombinant methods, independently of any immune response. An “antigen moiety” is a monovalent derivative of an antigen.

“Antibody library” refers to a repertoire or synthetic library of genes encoding antibodies or antibody fragments such as Fab, scFv, Fd, LC, VH, or VL, which is obtained from the natural ensemble, or “repertoire”, of antibodies present in a subject donor. The antibodies are typically obtained primarily from the cells of peripheral blood and spleen. In a some embodiments, human donors may be “non-immune”, i.e., not presenting with symptoms of infection.

“Synthetic antibody library” refers to a library of genes encoding one or more antibodies or antibody fragments such as Fab, scFv, Fd, LC, VH, or VL, in which one or more of the complementarity-determining regions (CDR) has been partially or fully randomized by oligonucleotide-directed mutagenesis. “Randomized” means that part or all of the sequence encoding the CDR has been replaced by sequence randomly encoding all twenty amino acids or some subset of the amino acids.

A “phosphoester moiety,” as used herein, refers to a divalent radical having the formula P(O)OR′. The symbol R′ represents a substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

An “affinity tag” is a functional moiety capable of specifically and non-covalently binding to a complimentary functional moiety (also referred to herein as an “affinity tag binder” to form an affinity tag-affinity tag binder pair). The affinity tag may be used to immobilize a compound onto a solid support. In some cases, the affinity tag may be a simple chemical functional group (e.g. biotin). Other possibilities include amino acids, poly(amino acid) tags, or full-length proteins, carbohydrates and nucleic acids, as detailed below. For instance, the affinity tag may be a polynucleotide which hybridizes to another polynucleotide serving as a functional group on the solid support. In some embodiments, where the solid support comprises a lipid bilayer or monolayer, then a membrane anchor is a suitable affinity tag. A variety of non-covalently binding affinity tags are useful including, for example, those based on ionic interactions, hydrogen bonding, hydrophobic interactions, hydrophilic interactions and hydrogen bonding interactions.

The term “isolated,” when applied to a nucleic acid or protein, denotes that the nucleic acid or protein is essentially free of other cellular components with which it is associated in the natural state. It may be in a homogeneous state, although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. In an exemplary embodiment, the nucleic acid or protein is purified when at least 85% pure. In another exemplary embodiment, the nucleic acid or protein is purified when at least 95% pure. In another exemplary embodiment, the nucleic acid or protein is purified when at least 99% pure.

A “sample” as used herein, refers to a representative part or a single item from a larger group chosen for analysis using the methods and/or systems of the present invention. A variety of samples may be analyzed using the present invention. Samples include environmental or biological materials derived from a bodily, cellular, viral and/or prion source. Some samples are derived from biological fluids such as saliva, blood and urine. In some embodiments, the biological fluids include whole cells, cellular organelles or cellular molecules such as a protein, protein fragment, peptide, carbohydrate or nucleic acid. The biological material can be endogenous or non-endogenous to the source. For example, in one embodiment, the biological material is a recombinant protein harvested from a bacteria and engineered using molecular cloning techniques (see generally, Sambrook et al, Molecular Cloning: A Laboratory Manual, 2d ed. (1989) Cold Spring Harbor Laboratory Press, Cold Spring Harbor, N.Y., which is incorporated herein by reference). In another embodiment, the sample comprises a chemically synthesized biological material such as a synthetic protein, protein fragment, peptide, carbohydrate or nucleic acid.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) as well as intervening sequences (introns) between individual coding segments (exons).

“Peptide” refers to a polymer in which the monomers are amino acids and are joined together through amide bonds, alternatively referred to as a “polypeptide.” The terms “peptide” and “polypeptide” encompass proteins. Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
Alanine (A), Glycine (G);
Aspartic acid (D), Glutamic acid (E);
Asparagine (N), Glutamine (Q);
Arginine (R), Lysine (K);
Isoleucine (I), Leucine (L), Methionine (M), Valine (V);
Phenylalanine (F), Tyrosine (Y), Tryptophan (W);
Serine (S), Threonine (T); and
Cysteine (C), Methionine (M)
   (see, e.g., Creighton, Proteins (1984)).

The term “cleaving” as used herein refers to the hydrolysis of at least one peptide bond within the amino acid chain of a peptide, polypeptide or protein.

“Complementarity-determining domains” or “CDR” refers to the hypervariable regions of V_{L} and V_{H}. The CDRs are the target protein-binding site of the antibody chains that harbors specificity for such target protein. There are three CDRs (CDR1-3, numbered sequentially from the N-terminus) in each human V_{L} or V_{H}, constituting about 15-20% of the variable domains. The CDRs are structurally complementary to the epitope of the target protein and are thus directly responsible for the binding specificity. The remaining stretches of the V_{L} or V_{H}, the so-called framework regions, exhibit less variation in amino acid sequence (Kuby, Immunology, 4^{th} ed., Chapter 4. W.H. Freeman & Co., New York, 2000).

The positions of the CDRs and framework regions are determined using various well known definitions in the art, e.g., Kabat, Chothia, international ImMunoGeneTics database (IMGT), and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al, Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

“Mutating” or “mutation” as used in the context of altering the enzymatic activity of a recombinant catalytic polypeptide refers to the deletion, insertion, or substitution of any nucleotide, by chemical, enzymatic, or any other means, in a nucleic acid encoding a recombinant catalytic polypeptide such that the amino acid sequence of the resulting polypeptide is altered at one or more amino acid residues.

### INTRODUCTION

The present invention provides halogen phosphonate monoester probes, immobilizing reagents, and antigen conjugates for use in novel methods of detecting, immobilizing, and producing proteolytic antibodies. The halogen phosphonate monoester probes, immobilizing reagents, and antigen conjugates of the present invention are capable of covalently binding a proteolytic antibody.

### I. Phosphonate Monoester Compositions

A. Halogen Phosphonate Monoester Probes

Halogen phosphonate monoester probes are compounds having a phosphoester moiety, wherein the central phosphorus of the phosphoester moiety is covalently bound, either directly or indirectly, to a halogen and a detectable moiety. In some embodiments, the halogen is directly covalently bound to the central phosphorus of the phosphoester moiety. The central phosphorus forms part of only one phosphoester moiety.

In an exemplary embodiment, the halogen phosphonate monoester probe has the formula:

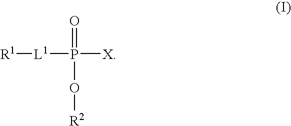

In Formula (I), the symbol X represents a halogen. In an exemplary embodiment, X is selected from F, Cl, Br, and I. In another exemplary embodiment, X is F.

L¹ is selected from a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. However, L¹ is not attached to the phosphorus via an oxygen heteroatom;

The symbol R¹ represents a detectable label. R² is selected from the group consisting of a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, L¹ is selected from a bond, substituted or unsubstituted C₁-C₅₀ alkylene, substituted or unsubstituted 2 to 50 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene. In other embodiments, L¹ is selected from a bond, substituted or unsubstituted C₁-C₄₀ alkylene, and substituted or unsubstituted 2 to 40 membered heteroalkylene.

L¹ may also be selected from the group consisting of a bond; unsubstituted C₁-C₄₀ alkylene; unsubstituted 2 to 40 membered heteroalkylene; and C₁-C₄₀ alkylene or 2 to 40 membered heteroalkylene substituted with a substituent (also referred to herein as an “L¹ substituent”). The L¹ substituent is independently selected from an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

In another exemplary embodiment, L¹ is a 2 to 40 membered heteroalkylene substituted with an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

R² may be selected from hydrogen, substituted or unsubstituted C₁-C₅₀ alkyl, substituted or unsubstituted 2 to 50 membered heteroalkyl, substituted or unsubstituted C₃-C₈ cycloalkyl, substituted or unsubstituted 3 to 8 membered heterocycloalkyl, substituted or unsubstituted aryl, or substituted or unsubstituted heteroaryl. R² may also be selected from hydrogen, substituted or unsubstituted C₁-C₂₀ alkyl, substituted or unsubstituted 2 to 20 membered heteroalkyl, substituted or unsubstituted C₃-C₈ cycloalkyl, substituted or unsubstituted 3 to 8 membered heterocycloalkyl, substituted or unsubstituted aryl, or substituted or unsubstituted heteroaryl. In another exemplary embodiment, R² is selected from hydrogen, substituted or unsubstituted C₁-C₁₀ alkyl, substituted or unsubstituted 2 to 10 membered heteroalkyl, substituted or unsubstituted C₃-C₈ cycloalkyl, substituted or unsubstituted 3 to 8 membered heterocycloalkyl, substituted or unsubstituted aryl, or substituted or unsubstituted heteroaryl.

In some embodiments, R² is selected from hydrogen; unsubstituted C₁-C₁₀ alkyl; unsubstituted 2 to 10 membered heteroalkyl; unsubstituted C₃-C₈ cycloalkyl; unsubstituted 3 to 8 membered heterocycloalkyl; unsubstituted aryl; unsubstituted heteroaryl; and C₁-C₁₀ alkyl, 2 to 10 membered heteroalkyl, C₃-C₈ cycloalkyl, 3 to 8 membered heterocycloalkyl, aryl, or heteroaryl substituted with a substituent (also referred to herein as an “R² substituent”). The “R² substituent” is independently selected from oxy, unsubstituted C₁-C₂₀ alkyl, unsubstituted 2 to 20 membered heteroalkyl, unsubstituted C₃-C₈ cycloalkyl, unsubstituted 3, to 8 membered heterocycloalkyl, unsubstituted aryl, and unsubstituted heteroaryl. In other embodiments, R² is unsubstituted C₁-C₁₀ alkyl.

The compounds of Formula (I) can be prepared by reacting the corresponding alcohol (e.g. a protonated phosphate wherein the halogen is replaced by a hydroxyl moiety) with a halogenating agent. Halogenating agents are well known in the art of general organic synthesis, and examples can be found in several organic synthesis textbooks (Buehler and Pearson, Survey of Organic Synthesis, John Wiley and Sons, New York (1977); Harrison and Harrison, Compendium of Organic Synthetic Methods, John Wiley and Sons, New York (1974)). For example, a fluoride can be added using the reagent DAST, a bromide by using a phosphorotribromide, or a chloride can be added using thionylchloride.

1. Detectable Labels

A detectable label is a group that is detectable at low concentrations, usually less than micromolar, possibly less than nanomolar, that can be readily distinguished from other molecules, due to differences in a molecular property (e.g. molecular weight, mass to charge ratio, redox potential, luminescence, electromagnetic properties, binding properties, and the like). A wide variety of detectable labels are useful in the present invention, including hapten labels (e.g. biotin, or labels used in conjunction with detectable antibodies such as horse radish peroxidase antibodies), mass tag labels (e.g. stable isotope labels), radioisotopic labels, metal chelate labels, luminescent labels (e.g. fluorescent, phosphorescent, and chemiluminescent labels, typically having quantum yield greater than 0.1), electroactive labels, enzyme modulator labels (e.g. coenzymes, organometallic catalysts), photosensitizer labels, and electron transfer labels.

Useful labels may be detected by spectroscopic, photochemical, biochemical, immunochemical, electrical, magnetic, electromagnetic, optical or chemical means. Exemplary labels include magnetic bead labels (e.g., Dynabeads™); fluorescent dye labels (e.g., fluorescein isothiocyanate, texas red, rhodamine, green fluorescent protein, and the like); radiolabels (e.g., H³, I¹²⁵, S³⁵, C¹⁴, or P³²); enzyme labels (e.g., horse radish peroxidase, alkaline phosphatase and others commonly used in an ELISA); colorimetric labels such as colloidal gold, silver, selenium, or other metals and metal sol labels (see U.S. Pat. No. 5,120,643, which is herein incorporated by reference in its entirety for all purposes), or colored glass or plastic (e.g., polystyrene, polypropylene, latex, etc.) bead labels; and carbon black labels. Patents teaching the use of such detectable labels include U.S. Pat. Nos. 3,817,837; 3,850,752; 3,939,350; 3,996,345; 4,277,437; 4,275,149; 4,366,241; 6,312,914; 5,990,479; 6,207,392; 6,423,551; 6,251,303; 6,306,610; 6,322,901; 6,319,426; 6,326,144; and 6,444,143, which are herein incorporated by reference in their entirety for all purposes.

In an exemplary embodiment, the detectable label is selected from biotin, deiminobiotin, dethiobiotin, vicinal diols, such as 1,2-dihydroxyethane, 1,2-dihydroxycyclohexane, etc., digoxigenin, maltose, oligohistidine, glutathione, 2,4-dinitrobenzene, phenylarsenate, ssDNA, dsDNA, a peptide, a polypeptide, a protein a metal chelate, a saccharide, rhodamine or fluorescein, or any hapten to which an antibody can be generated.

A variety of fluorescent detectable labels may be employed. Many such labels are commercially available from, for example, the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GEBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired fluorescent label.

The detectable labels may be covalently attached to the halogen phosphonate monoester probe using a reactive functional group, which can be located at any appropriate position. When the reactive group is attached to an alkyl, or substituted alkyl chain tethered to an aryl nucleus, the reactive group may be located at a terminal position of an alkyl chain. Reactive groups and classes of reactions useful in practicing the present invention are generally those that are well known in the art of bioconjugate chemistry. Currently favored classes of reactions available with reactive known reactive groups are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides or reacted with acyl halides;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds; and

(k) phosphoramidites and other standard functional groups useful in nucleic acid synthesis.

The reactive functional groups can be chosen such that they do not participate in, or interfere with, the crosslinking reactions disclosed herein. Alternatively, a reactive functional group can be protected from participating in the crosslinking reaction by the presence of a protecting group. Those of skill in the art will understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

Linkers may also be employed to attach the detectable labels to the halogen phosphonate monoester probe. Linkers may include reactive groups at the point of attachment to the detectable label and/or the mobile detectable analyte binding reagents. Any appropriate linker may be used in the present invention, including substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. Other useful linkers include those having a polyester backbone (e.g. polyethylene glycol), nucleic acid backbones, amino acid backbones, and derivatives thereof. A wide variety of useful linkers are commercially available (e.g. polyethylene glycol based linkers such as those available from Nektar, Inc. of Huntsville, Ala.).

B. Halogen Phosphonate Monoester Immobilizing Reagents

Halogen phosphonate monoester immobilizing reagents are compounds having a phosphoester moiety, wherein the central phosphorus of the phosphoester moiety is covalently bound, either directly or indirectly, to a halogen and an immobilizing moiety or solid support. In some embodiments, the halogen is directly covalently bound to the central phosphorus of the phosphoester moiety. The central phosphorus forms part of only one phosphoester moiety.

In an exemplary embodiment, the halogen phosphonate monoester immobilizing reagent has the formula:

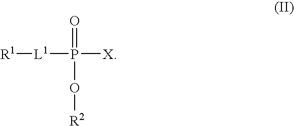

In Formula (II), L¹, R², and X are as defined above in the discussion of Formula (I). R¹ is an immobilizing moiety or solid support.

1. Immobilizing Moieties

An immobilizing moiety is a moiety capable of binding to a solid support covalently or non-covalently thereby attaching (also referred to herein as “immobilizing”) the immobilizing moiety (with or without the proteolytic antibody) to the solid support. Typically, the immobilizing moiety will bind to complimentary solid support. As used herein, a “complimentary solid support” is a solid support having a binding moiety (e.g. an affinity tag binder to reactive group) that binds to the immobilizing moiety (e.g. an affinity tag or a crosslinking group). A wide variety of immobilizing moieties are useful in the present invention. In an exemplary embodiment, the immobilizing moiety is an affinity tag or crosslinking group. The affinity tag and/or crosslinking group may form half of a binder pair. For example, where the halogen phosphonate monoester immobilizing reagent includes an affinity tag, the solid support to which the phosphonate monoester immobilizing reagent binds will include an affinity tag binder, to which the affinity tag binds, thereby forming an affinity tag-affinity tag binder pair. Likewise, where the halogen phosphonate monoester immobilizing reagent includes a crosslinking group, the solid support includes a reactive group to which the crosslinking group covalently binds thereby forming a crosslinking group-reactive group pair.

Any appropriate affinity tag may be used in the present invention. Affinity tags are well known in the art and include, for example, biotin, deiminobiotin, dethiobiotin, vicinal diol, digoxigenin, maltose, oligohistidine, glutathione, 2,4-dinitrobenzene, phenylarsenate, ssDNA, dsDNA, polyhistidine, a hapten, T7 tag, S tag, His tag, GST tag, PKA tag, HA tag, c-Myc tag, Trx tag, Hsv tag, CBD tag, Dsb tag, pelB/ompT, KSI, MBP tag, VSV-G tag, β-Gal tag, GFP tag, V5 epitope tag, and FLAG epitope tag (Eastman Kodak Co., Rochester, N.Y.). In an exemplary embodiment, the complimentary solid support comprises a complimentary affinity tag binder that binds to the affinity tag.

In some embodiments, the immobilizing moiety is a crosslinking group. A crosslinking group is a chemical moiety capable of covalently linking the halogen phosphonate monoester immobilizing reagent to a solid support. A wide variety of chemical crosslinking groups are useful in the present invention, including those reactive functional groups discussed above in the context of detectable label attachment to halogen phosphonate monoester probes. See Greene et al., PROTECTIVE GROUPS IN ORGANIC SYNTHESIS, John Wiley & Sons, New York, 1991. Other useful covalent linkages may be found, for example, in texts relating to the art of solid phase synthesis of biomolecules such as peptides, polypeptide, proteins, nucleic acids and carbohydrates (see, e.g., Eckstein et al, Oligonucleotides and Analogues: A Practical Approach, (1991); Stewart et al., Solid Phase Peptide Synthesis, 2nd Ed., (1984); Seeberger, Solid support oligosaccharide synthesis and combinatorial carbohydrate libraries (2001)). In some embodiments, intein-mediated protein ligation may be used to attach the halogen phosphonate monoester immobilizing reagent to a solid support (Mathys, et al., Gene 231:1-13, 1999; Evans, et al., Protein Science 7:2256-2264, 1998).

2. Solid Supports

The term “solid support” refers to a material in the solid-phase that is capable of interacting with reagents in the liquid phase. Solid supports can be derivatized with reactive functional groups (such as those described above in the context of detectable label attachment to halogen phosphonate monoester probes), affinity tag binders (i.e. those groups that specifically bind non-covalently to affinity tags to from an affinity tag-affinity tag binder pair), biomolecules (including enzymes, peptides, oligonucleotides and polynucleotides), and the like. Typically, the solid support is a complimentary solid support that includes a binding group or reactive group that is complementary to (i.e. binds to) an immobilizing moiety.

A wide array of solid supports are useful in the present invention. Solid supports are typically composed of insoluble materials. In some embodiments, the supports have a rigid or semi-rigid character, and may be any shape, e.g. spherical, as in beads, rectangular, irregular particles, resins, gels, microspheres, or substantially flat as in a microchip. Arrays of physically separate regions may be present on the support with, for example, wells, raised regions, dimples, pins, trenches, rods, pins, inner or outer walls of cylinders, and the like.

Preferred support materials include agarose, polyacrylamide, magnetic beads (Stamm, 1995), polystyrene (Andrus, 1993), controlled-pore-glass (Caruthers, 1984), polyacrylate hydroxethylmethacrylate, polyamide, polyethylene, polyethyleneoxy, or copolymers and grafts of such. The hydrophilic nature of the polyethyleneoxy groups promotes rapid kinetics and binding when aqueous solvents are used. Other solid supports include small particles, membranes, frits, non-porous surfaces, addressable arrays, vectors, plasmids, or polynucleotide-immobilizing media. Additionally, fullerenes may be used as a solid support, as well as derivatized fullerenes such as gadolinium fullerenes which contain paramagnetic properties.

In an exemplary embodiment, the solid support includes those composed of polystyrene, polyethylene, polyacrylamide, polypropylene, polyamide, Merrifield resin, sepharose, agarose, polystyrene, polydivinylbenzene, cellulose, alginic acid, chitosan, chitin, polystyrene-benzhydrylamine resin, an acrylic ester polymer, a lactic acid polymer, silica, silica gel, amino-functionalized silica gel, alumina, clay, zeolite, glass, controlled pore glass, or montmorillonite.

C. Halogen Phosphonate Monoester Antigen Conjugates

Halogen phosphonate monoester antigen conjugates are compounds having a phosphoester moiety, wherein the central phosphorus of the phosphoester moiety is covalently bound, either directly or indirectly, to a protein antigen moiety. In some embodiments, the halogen is directly covalently bound to the central phosphorus atom. The central phosphorus forms part of only one phosphoester moiety.

In an exemplary embodiment, the halogen phosphonate monoester antigen conjugate has the formula

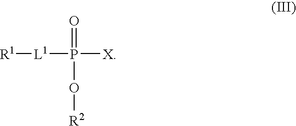

In Formula (III), L¹, R², and X are as defined above in the discussion of Formula (I).

R¹ is an antigen moiety. In some embodiments, R¹ is a non-hydrolytic polypeptide moiety (i.e. a monovalent derivative of polypeptide that does not catalyze the hydrolysis of a amide linkage in a peptide or protein). In other embodiments, R¹ is selected from the group of a growth factor, cell surface receptor, cytokine, or immunoglobulin. R¹ may also be selected from TNFα, vascular endothelial growth factor, interferon-γ, and CD20. Where the antigen moiety is selected from a group of proteins, is understood that the proteins are moieties, wherein the protein is monovalent radical derivative of the named protein attached to the remainder of the phosphonate monoester antigen conjugate. Other exemplary protein antigen moieties are discussed below in detail.

1. Antigen Moieties

An antigen moiety is a monovalent radical derivative of an antigen useful in illiciting an immune response in a subject. In some embodiments, the antigen moiety is a protein antigen moiety, such as a therapeutic or prophylactic protein moiety, or portion thereof.

Examples of therapeutic or prophylactic proteins useful as protein antigen moieties include, for example, hormones (e.g. insulin, glucogon-like peptide 1 and parathyroid hormone), antibodies, inhibitors, growth factors, postridical hormones, nerve growth hormones, blood clotting factors (e.g. Factor VIII, TPA), adhesion molecules, bone morphogenic proteins, lectins, trophic factors, cytokines (e.g. TGF-β, IL-2, IL-4, α-IFN, .β-IFN, γ-IFN, TNF, IL-6, IL-8, lymphotoxin, IL-5, migration inhibition factor, GMCSF, IL-7, IL-3, monocyte-macrophage colony stimulating factors, granulocyte colony stimulating factors), multidrug resistance proteins, other lymphokines, toxoids, erythropoietin, amylin, dornase-α, α-1-antitripsin, human growth hormones, nerve growth hormones, bone morphogenic proteins, urease, toxins or toxoids (e.g. anthrax toxin, botulinum neurotoxin), fertility hormones (e.g. FSH and LSH).

Useful therapeutic proteins include leukocyte markers, such as CD2, CD3, CD4, CD5, CD6, CD7, CD8, CD11a, CD11b, CD11c, CD13, CD14, CD18, CD19, CE20, CD22, CD23, CD27 (and its ligand), CD28 (and its ligands B7.1, B7.2, B7.3), CD29 (and its ligands), CD30 (and its ligand), CD40 (and its ligand gp39), CD44, CD45 (and isoforms), Cdw52 (Campath antigen), CD56, CD58, CD69, CD72, CTLA-4, and LFA-1. Also included are TCR histocompatibility antigens (e.g. MHC class I or II antigens, the Lewis Y antigens, SLex, SLey, SLea and SLeb), integrins (e.g. VLA-1, VLA-2, VLA-3, VLA-4, VLA-5, VLA-6 and LFA-1), adhesion molecules (e.g. Mac-1 and p150,95), and selectins (e.g. L-selectin, P-selectin, E-selectin and their counter receptors VCAM-1, ICAM-1, ICAM-2 and LFA-3).

Interleukins may also be useful therapeutic proteins, including, IL-1, IL-2, IL-3, IL-4, IL-5, IL-6, IL-7, IL-8, IL-10, IL-11, IL-12, IL-13, IL-14 and IL-15. In some embodiments, interleukin receptors are used as the antigen moiety, including, for example, IL-1R, IL-2R, IL-4R, IL-5R, IL-6R, IL-7R, IL-8R, IL-10R, IL-11R, IL-12R, IL-13R, IL-14R and IL-15R. In other embodiments, the antigen moiety is a chemokine (e.g. PF4, RANTES, MIP1α, MCP1, NAP-2, Groα, Groβ and IL-8), a growth factor (e.g. TNFα, TGFβ, TSH, VEGF/VPF, PTHrP, EGF family, EGF, PDGF family, endothelin and gastrin releasing peptide (GRP)), or a growth factor receptor (e.g. TNFαR, RGFβR, TSHR, VEGFR/VPFR, FGFR, EGFR, PTHrPR, PDGFR family, EPO-R). Other useful receptors include, for example GCSF-R and other hematopoietic receptors, interferon receptors (e.g. IFNαR, IFNβR and IFNγR), and Igs and their receptors (e.g. IgE, FceRI and FceRII).

The antigen moiety may also be a blood factor (e.g. complement C3b, complement C5a, complement C5b-9, Rh factor, fibrinogen, fibrin and myelin associated growth inhibitor). The non-hydrolytic protein component of this invention may be any natural, synthetic or recombinant protein antigen including, for example, tetanus toxoid, diptheria toxoid, viral surface proteins (e.g. CMV glycoproteins B, H and gCIII), HIV-1 envelope glycoproteins, RSV envelope glycoproteins, HSV envelope glycoproteins, EBV envelope glycoproteins, VZV envelope glycoproteins, HPV envelope glycoproteins, Influenza virus glycoproteins, Hepatitis family surface antigens; viral structural proteins, viral enzymes, parasite proteins, parasite glycoproteins, parasite enzymes and bacterial proteins. Also included are tumor antigens, such as her2-neu, mucin, CEA and endosialin.

Antigen moieties may also include allergens, such as house dust mite antigen, 1ol p1 (grass) antigens or urushiol. In some embodiments, the antigen moiety is a toxin, such as pseudomonas endotoxin, osteopontin/uropontin, snake venom, or bee venom. Also included are glycoprotein tumor-associated antigens, such as carcinoembryonic antigen (CEA), human mucins, her-2/neu and prostate-specific antigen (PSA) (R. A. Henderson and O. J. Finn, Advances in Immunology, 62, pp. 217-56 (1996)).

The antigen moiety is typically covalently attached to the remainder of the halogen phosphonate monoester antigen conjugate. Thus, attachment may be achieved through an antigen reactive functional group. Useful antigen reactive functional groups may be selected from, but are not limited to, groups that will react directly with an amine group (e.g. lysine epsilon amine group, a terminal amine group in a peptide, or a sulfhydryl group such as a cysteine sulfhydryl group commonly found on a protein or other biological molecule). Examples of such protein reactive groups include active halogen-containing groups such as chloromethylphenyl groups, chloromethylcarbonyl groups, and iodomethylcarbonyl groups; activated 2-leaving-group-substituted ethylsulfonyl and ethylcarbonyl groups such as 2-chloroethylsulfonyl groups and 2-chloroethylcarbonyl groups; vinylsulfonyl groups; vinylcarbonyl groups; oxiranyl groups; isocyanato groups; isothiocyanato groups; aldehydro groups; aziridyl groups; succinimidoxycarbonyl groups; activated acyl groups such as carboxylic acid halide groups; anhydride groups; thioester groups; carbonates such as nitrophenylcarbonates; sulfonic acid esters; phosphoramidates; cyanuric monochlorides and cyanuric dichlorides; and other groups known to be useful in conventional photographic gelatin hardening agents. Other useful antigen reactive functional groups are chosen from the reactive functional groups discussed above in the context of detectable label attachment to halogen phosphonate probes.

The above listed protein reactive groups can react with a protein or other biological molecule which is chemically modified to contain reactive amine groups and sulfhydryl groups. Amine groups may be introduced by well known techniques such as, for example, nitration of a phenyl group followed by reduction, by conversion of a primary amide to an amine with nitrous acid, by conversion of a hydroxyl group of an alcohol into a sulfonic acid ester followed by displacement with an azide group and subsequent reduction to an amine, and the like. Sulfhydryl groups can be introduced by well known techniques such as, for example, by conversion of a hydroxyl group of an alcohol into a sulfonic acid ester followed by displacement with sodium sulfide, by dehydrative amide bond formation between an amine group of a protein and a carboxylic acid group of an acetylated cysteine using a carbodiimide reagent followed by treatment with hydroxylamine, and the like.

Where a protein or other biological molecule is chemically modified such as by partial oxidation to introduce an aldehyde group or a carboxylic acid group, exemplary antigen reactive functional groups may be selected from amino, aminoalkyl, aminoaryl, alkylamino, arylamino, hydrazino, alkylhydrazino, arylhydrazino, carbazido, semicarbazido, thiocarbazido, thiosemicarbazido, sulfhydryl, sulfhydrylalkyl, sulfhydrylaryl, hydroxy, carboxy, carboxyalkyl and carboxyaryl.

An additional preferred antigen reactive functional group may bind to or include a residue of a bifunctional crosslinking agent. Useful bifunctional crosslinking agents may react with a functional group such as amine, sulfhydryl, carboxylic acid, and aldehyde groups. Exemplary bifunctional crosslinking reagents include difunctional gelatin hardeners, bisepoxides and bisisocyanates.

The binding of the antigen to the remainder of the halogen phosphonate monoester antigen conjugate may be facilitated with a consumable catalyst, such as carbodiimide and carbamoylonium as disclosed in U.S. Pat. No. 4,421,847 and the dication ethers of U.S. Pat. No. 4,877,724, both of which are herein incorporated herein by reference in their entirety for all purposes.

Additional antigen reactive functional groups include, for example, semicarbazido; thiocarbazido; thiosemicarbazido; isocyanato, isothiocyanato, active esters (e.g. succinimidoxycarbonyl), active anhydrides, mixed anhydrides, and azido. In an exemplary embodiment, the antigen reactive functional group is a sulfhydryl, amino, isothiocyanato or arylcarbonatoalkyl.

Other exemplary antigen reactive functional groups include amidatoalkyloxy; hydrazidoalkyloxy; azidocarbonylalkyloxy; and aryloxycarbonyloxyalkyloxy, formylalkyl, aminoalkyl, vinyl sulfonylalkyloxy, active carbonates (e.g. arylcarbonatoaryl, alkylcarbonatoaryl, arylcarbonatoalkyl, alkylcarbonatoalkyl), iodoalkylcarbonylamino, amidatoalkylamino, and amidatoarylalkylamino. The alkylene groups of these functional groups may contain from 1 to 10 carbon atoms. In related embodiments, the alkylene group contains from 2 to 10 carbons.

In an exemplary embodiment, the antigen reactive functional group includes an alkylcarbonatoalkyl. The alkylene group may contain from 2 to 10 carbon atoms. The aryl group may be a six membered ring containing electron withdrawing substituents such as, for example, nitro and halogen, and optionally containing water solubilizing groups. Exemplary water solubilizing groups include sulfonate salt, sulfhydryl, thioalkylcarbonylaminoalkyloxy and sulfhydrylalkyl. In some embodiments, the alkyl group of the sulfhydrylalkyl contains from 1 to 10 carbon atoms. In another embodiment, the alkylene group of the thioalkylcarbonyl portion of the thioalkylcarbonylaminoalkyloxy may contain from 1 to 10 carbon atoms, and the alkylene group of the aminoalkyloxy portion may contain from 2 to 10 carbon atoms.

In some embodiments, the antigen reactive functional group is vinyl sulfonylalkylpoly(oxyalkyl)oxy. The alkylene group of the sulfonylalkyl portion may contain from 2 to 10 carbon atoms and/or the alkylene group of the polyoxyalkyl portion may contain from 2 to 10 carbon atoms. Exemplary vinyl sulfonylalkylpoly(oxyalkyl)oxy functional groups include those compounds where a poly(oxyalkyl) portion includes a poly(oxyethylene) group or a poly(oxyethylene)-co-poly(oxypropylene) copolymer group. The polymer may from 2 to about 100 monomeric oxyalkylene units.

Another useful antigen reactive functional group is aryloxycarbonyl(polyoxyalkyl)oxy. The alkylene group of the polyoxyalkyl portion may contain from 2 to 10 carbon atoms. The poly(oxyalkyl) portion may include a poly(oxyethylene) group or a poly(oxyethylene)-co-poly(oxypropylene) copolymer group. The polymer may contain from 2 to about 100 monomeric oxyalkylene units;

Triazines may also be used as an antigen reactive functional groups. Exemplary triazines include 4,6-dichloro-2-triazinylamino, 4,6-dichloro-2-triazinyloxy, 4,6-dichlorotriazinyl-2-oxy(polyalkyloxy), 4-alkoxy-6-chloro-2-triazinyloxy, and 4-alkoxy-6-chloro-2-triazinyl(polyoxyalkyl)oxy). The alkyl groups of the alkoxy portions may each contain from 2 to 10 carbon atom. The alkylene groups of the polyoxyalkyl portions may each contain from 2 to 10 carbon atoms. Exemplary poly(oxyalkyl) portions may include a poly(oxyethylene) group or a poly(oxyethylene)-co-poly(oxypropylene)copolymer group, in which the polymer contains from 2 to about 100 monomeric oxyalkylene units.

In some embodiments, the antigen reactive functional group is maleimidoalkylcarbonyl-aminoalkyloxy. The alkylene group of the maleimidoalkylcarbonyl portion may contain from 1 to 10 carbon atoms. The alkylene group of the aminoalkyloxy portion may contain from 2 to 10 carbon atoms.

### II. Methods

A. Detecting the Presence of a Proteolytic Antibody

In another aspect, methods of detecting the presence of a proteolytic antibody using the halogen phosphonate monoester probe of the present invention are provided. The method may include contacting a proteolytic antibody with a halogen phosphonate monoester probe. As described above, the halogen phosphonate monoester probe includes a detectable label. The halogen phosphonate monoester probe is allowed to covalently bind to the proteolytic antibody. Thus, a proteolytic antibody-phosphonate conjugate is formed. Finally, the detectable label is detected thereby detecting the presence of the proteolytic antibody.

In an exemplary embodiment, the halogen phosphonate monoester probe is the probe of Formula (I), as described above.

The proteolytic antibody may be present in a sample, as defined above. The sample may include a plurality of additional antibodies (e.g. additional proteolytic and/or non-proteolytic antibodies). The proteolytic antibody may also form part of an antibody library or synthetic antibody library, as defined above. The library may include additional proteolytic and/or non-proteolytic antibodies.

In some embodiments, the proteolytic antibody is immobilized on a solid support. Useful solid supports are discussed above in the context of halogen phosphonate monoester immobilizing moieties. Thus, the halogen phosphonate monoester probe may be used to detect the presence of a proteolytic antibody on a solid support (such as a biochip) containing an antibody library or synthetic antibody library. Any appropriate method may be used to immobilize the proteolytic antibody, including the use of reactive functional groups or general antibody Fc binders attached to the solid support (e.g. Protein A, Protein G, etc.).

Proteolytic antibodies may be derived from a number of sources including animals and cell cultures. In some embodiments, the proteolytic antibodies are recombinantly produced through display technologies such as phage, ribosome, RNA, or plasmid display. In other embodiments, the proteolytic antibodies are derived from the serum of an animal. Such an animal may be immunized or unimmunized. Where the proteolytic antibody is present in a sample, the sample may be derived from a bodily, cellular, viral and/or prion source. In a related embodiment, the sample is derived from biological fluids such as saliva, blood and urine. Appropriate biological fluids include, for example, whole cells, cellular organelles or cellular molecules such as a protein, protein fragment, peptide, carbohydrate or nucleic acid. In other embodiments, the sample is derived from a human source.

Proteolytic antibodies can be identified by their ability to bind to a halogen phosphonate monoester probe. The presence of an antibody bound to a halogen phosphonate monoester probe can be detected by mixing the halogen phosphonate monoester probe with an antibody or mixture of antibodies in an appropriate buffer (e.g. Tris Cl, PIPES, HEPES, and the like).

Means of detecting detectable labels are well known to those of skill in the art. Thus, for example, radiolabels may be detected using photographic film or scintillation counters, and fluorescent markers may be detected using a photodetector to detect emitted illumination. Enzymatic labels are typically detected by providing the enzyme with a substrate and detecting the reaction product produced by the action of the enzyme on the substrate, and calorimetric labels are detected by simply visualizing the colored label. In some embodiments, detection of the detectable label includes quantification of the detectable label, which may be correlated to the quantify the proteolytic antibody present.

The methods of detecting a proteolytic antibody (as well as methods of immobilizing proteolytic antibodies and/or producing proteolytic antibodies in a subject, both of which are described in more detail below) may be combined with any appropriate detection and/or purification methodology. Exemplary detection and/or purification methodologies may include the use of standard protein purification methods, such as salt precipitation and solvent precipitation; methods utilizing the difference in molecular weight such as dialysis, ultra-filtration, gel-filtration, and SDS-polyacrylamide gel electrophoresis; methods utilizing a difference in electrical charge such as ion-exchange column chromatography, methods utilizing specific affinity such as affinity chromatography; methods utilizing a difference in hydrophobicity such as reverse-phase high performance liquid chromatography; and methods utilizing a difference in isoelectric point, such as isoelectric focusing electrophoresis. Additional visualization and/or quantification of the isolated or non-isolated proteolytic antibody-phosphonate conjugate may be accomplished using any appropriate technique, including the use of dyes not covalently bound to the phosphonate (e.g. protein dyes such as Commassie Blue). Where gel purification is used, a band containing the proteolytic antibody-phosphonate conjugate can be isolated by excision from the gel using procedures well known to those of skill in molecular biology or biochemistry.

B. Immobilizing a Proteolytic Antibody

In another aspect, methods for immobilizing a proteolytic antibody using the halogen phosphonate monoester immobilizing reagent of the present invention are provided. The method includes contacting a proteolytic antibody with a halogen phosphonate monoester immobilizing reagent. The halogen phosphonate monoester immobilizing reagent includes a solid support or immobilizing moiety, as described above (e.g. a biotin moiety that binds to an avidin or streptavidin on the solid support). The halogen phosphonate monoester immobilizing reagent is allowed to covalently bind to the proteolytic antibody thereby immobilizing the proteolytic antibody. Thus, an immobilized proteolytic antibody-phosphonate conjugate is produced. In some embodiments, the proteolytic antibody forms part of a sample.

Where the halogen phosphonate monoester immobilizing reagent includes an immobilizing moiety, the immobilizing moiety is allowed to bind to a complimentary solid support. As used herein, a “complimentary solid support” is a solid support having a binding moiety that binds to the immobilizing moiety. In an exemplary embodiment, the immobilizing moiety is selected from an affinity tag or a crosslinking group, as described above. In another exemplary embodiment, where the immobilizing moiety is an affinity tag, the complimentary solid support includes an affinity tag binder, as described above. In another exemplary embodiment, where the immobilizing moiety is a crosslinking group, the solid complimentary support includes a crosslinking group.

In some embodiments, the method further includes detecting the immobilized proteolytic antibody-phosphonate conjugate thereby detecting the presence of the proteolytic antibody. The immobilized proteolytic antibody-phosphonate conjugate may be detected by any appropriate means known in the art, including the use of detectable secondary antibodies (e.g. HRP conjugated antibody).

In an exemplary embodiment, the immobilized proteolytic antibody-phosphonate conjugate is separated from any unbound antibody present. The unbound antibody may be present in a sample. Separation may be achieved using one or more purification steps to separate unbound antibody form the solid support. For example, the purification step may include washing the solid support containing the immobilized proteolytic antibody with an appropriate solvent (e.g. aqueous solutions). Typically, the solvent is selected such that the proteolytic antibody remains immobilized during and after application of the solvent. selection of the appropriate solvent is well within the capabilities of those skilled in the art. In some embodiments, the solid support is a gel (e.g. polyacrylamide, agarose, and the like), wherein the antibody is separated from unbound probe by the use of an electric current.

Isolation may also be accomplished by incubating a sample containing proteolytic antibody with the probe, then contacting the sample with the solid support to immobilizes the proteolytic antibody. Any antibodies in the sample that are not immobilized are removed using any appropriate technique (e.g. by centrifugation and removal of the supernatant, or by discarding the flow through fraction in chromatography). The proteolytic antibody may be eluted from the solid support through methods well known in the art (e.g. with biotin, or other reagent competitive with the ligand).

The proteolytic antibody to be immobilized may be present in a sample containing a mixture of antibodies (e.g. additional proteolytic and/or non-proteolytic antibodies).

C. Producing a Proteolytic Antibody in a Subject

In another aspect, methods for producing a proteolytic antibody in a subject using the halogen phosphonate monoester antigen conjugate of the present invention are provided. The method includes administering a halogen phosphonate monoester antigen conjugate to the subject. The subject is allowed to produce proteolytic antibodies against the halogen phosphonate monoester antigen conjugate. Finally, the proteolytic antibodies are isolated from the subject, thereby producing the proteolytic antibody.

Typically, the antigen moieties are useful in directing the production of proteolytic antibodies. For example, a halogen phosphonate monoester antigen conjugate can bind specifically to an immunoglobulin comprising a catalytic dyad or triad, thus stimulating a B-cell expressing such an immunoglobulin to expand. Thus, in some embodiments, conjugation of a halogen phosphonate monoester to a therapeutic or prophylactic protein antigen is valuable in producing vaccines or induction of antibody therapeutics.

Techniques for administration of the halogen phosphonate monoester antigen conjugate are conventional and involve suitable immunization protocols and formulations which will depend on the nature of the antigen per se. For example, the antigen may be provided with a carrier to enhance its immunogenicity, include formulations which contain adjuvants, administer multiple injections, and/or vary the route of the immunization, etc. Such techniques are standard and optimization of them will depend on the characteristics of the particular antigen for which immunospecific reagents are desired. Such optimization is within the capabilities of those skilled in the art.

Polyclonal antibodies produced by the subject and secreted into the bloodstream can be recovered using known techniques. Purified forms of these antibodies may be readily prepared by standard purification techniques (e.g. affinity chromatography with Protein A, Protein G, anti-immunoglobulin, or the antigen itself). The success of immunization may be monitored by analyzing the antibody levels with respect to the antigen in serum using standard techniques such as ELISA, RIA and the like.

For some applications, only the variable regions of the antibodies might be used. Treating the polyclonal antiserum with suitable reagents so as to generate Fab′, Fab, or F(ab″)₂ fragments results in compositions lacking the entire antibody constant regions. Such fragments are useful, for example, in isolation or identification of covalently binding antibodies or catalytic antibodies.

Alternatively, immunoglobulins and analogs with desired characteristics can be generated from immortalized B cells derived from transgenic animals or from the rearranged genes provided by these animals in response to immunization. Thus, as an alternative to harvesting the antibodies directly from an organism, the B cells can be obtained (e.g. from the spleen, the peripheral blood lymphocytes, and/or lymph nodes) and immortalized using any of a variety of techniques, most commonly using the fusion methods described by Kohler and Milstein (Nature 245: 495 (1975)). The resulting hybridomas (or otherwise immortalized B cells) can then be cultured as single colonies and screened for secretion of antibodies of the desired specificity.

The screen may include a confirmation of the fully human character of the antibody. For example, as described in the examples below, a sandwich ELISA wherein the monoclonal in the hybridoma supernatant is bound both to antigen and to an antihuman constant region may be employed. After the appropriate hybridomas are selected, the desired antibodies may be recovered using conventional techniques. The hybridomas may be prepared in quantity by culturing the immortalized B cells using conventional methods, either in vitro or in vivo, to produce ascites fluid. Facile purification is possible as each immortalized colony will secrete only a single type of antibody. Standard purification techniques to isolate the antibody from other proteins in the culture medium may be employed.

As an alternative to obtaining human immunoglobulins directly from the culture of immortalized B cells derived from the animal, the immortalized cells can be used as a source of rearranged heavy chain and light chain loci for subsequent expression and/or genetic manipulation. Rearranged antibody genes can be reverse transcribed from appropriate mRNAs to produce cDNA. If desired, the heavy chain constant region can be exchanged for that of a different isotype or eliminated altogether. The variable regions can be linked to encode single chain Fv regions. Multiple Fv regions can be linked to confer binding ability to more than one target, or chimeric heavy and light chain combinations can be employed. Once the genetic material is available, design of analogs as described above (retain both their ability to bind the desired target and their human characteristics) is straightforward.

Once the appropriate genetic material is obtained (and, if desired, modified to encode a sequence analog), the coding sequences can be inserted into expression systems contained on vectors which can be transfected into standard recombinant host cells. The coding sequences may include those that encode, at a minimum, the variable regions of the human heavy and light chain. As described below, a variety of such host cells may be used. Typically, mammalian cells are used. Exemplary mammalian cell lines useful for this purpose include CHO cells, 293 cells, or NSO cells.

The production of the antibody or analog is then undertaken by culturing the modified recombinant host under culture conditions appropriate for the growth of the host cells and the expression of the coding sequences. The antibodies are then recovered from the culture. The expression systems may be designed to include signal peptides so that the resulting antibodies are secreted into the medium. However, intracellular production is also possible.

Phage display may also be employed in the current methods. The technology of phage-displayed antibodies is now well-established (Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., Immunol Today 17:453-455 (1996); Barbas et al., Trends in Biotechnol. 14:230-234 (1996); Winter et al., Ann. Rev. Immunol. 433-455 (1994)), and techniques and protocols required to generate, propagate, screen (pan), and use the antibody fragments from such libraries have recently been compiled (Phage Display of Peptides and Proteins: A Laboratory Manual, Kay, B K, Winter, J, McCafferty, J. (eds.), San Diego: Academic Press, Inc. 1996 (hereinafter, “Phage Display Manual”); Abelson et al. (eds.), Combinatorial Chemistry, Methods in Enzymology vol. 267, Academic Press (May 1996)). The basic details of library construction, screening and expression need not, therefore, be repeated here, as they are well within the knowledge of the skilled molecular biologist.

In addition, commercial kits are now available that allow the construction, propagation, and screening of phage display antibody libraries. Among these is the Recombinant Phage Antibody System (RPAS) available from Pharmacia Biotech (Amersham Pharmacia Biotech, catalogue number 27-9400-01). The RPAS system allows the expression of scFvs either as fusions to the pIII protein of filamentous phage for screening and propagation, or as soluble scFv antibody fragments for purposes of protein production. The form of the antibody fragment is determined by the choice of the chosen E. coli host strain. In addition, the RPAS system expresses the scFvs in tandem with an expression “tag” (“E” “tag”) which can be used for affinity purification or ELISA detection of the soluble scFvs.

### III. Proteolytic Antibody Immobilization Systems

In another aspect, the present invention provides a proteolytic antibody immobilization system. The system includes a halogen phosphonate monoester immobilizing reagent and a solid support (e.g. a complimentary solid support). Halogen phosphonate monoester immobilizing reagents and solid supports are reviewed in detail above.

In an exemplary embodiment, the immobilizing moiety is selected from an affinity tag or a crosslinking group. In another exemplary embodiment, where the immobilizing moiety is an affinity tag, the solid support includes an affinity tag binder, as described above. In another exemplary embodiment, where the immobilizing moiety is a crosslinking group, the solid support includes a crosslinking group.

### IV. Assays

One of skill in the art will immediately recognize that a wide variety of assays are available for identifying useful halogen phosphonate monoester probes, immobilizing reagents, and antigen conjugates. Useful assays may include methods of determining whether halogen phosphonate monoester probes, immobilizing reagents, and antigen conjugates are capable of binding to proteolytic antibodies.

As discussed above, analyses of catalytic antibody components have shown that enzymatic activity often resides in the light chains, and antibody light chains isolated from multiple myeloma patients frequently demonstrate proteolytic activity. See Paul, et al., Science, 244:1158-1162 (1989). Moreover, studies have provided evidence connecting proteolytic antibodies and serine proteases. See Gao et al., J. Bio. Chem., 269:32389-32393 (1994); Gao et al., J. Bio. Chem., 253:658-664 (1995)); (Paul, et al. J. Biol. Chem. 276: 28314-28320 (2001); Planque, et al. J. Biol. Chem. 278: 20436-20443 (2003); Paul, et al. J. Biol. Chem. 276: 28314-28320 (2001); (Rader, et al. Proc Natl Acad Sci USA 100: 5396-400 (2003); Chmura, et al. Proc Natl Acad Sci USA 98: 8480-4 (2001); Liu, et al. Proc. Natl. Acad. Sci. 96: 14694-14699 (1999).

Serine proteases are well characterized in the art of protease mechanistic action. Therefore, a wide array of assays are known in the art for identifying compounds that covalently bind to serine protease, including components of the catalytic triad or dyad. As demonstrated in the referenced studies above, these methods may also be used to identify compounds that covalently bind to proteolytic antibodies, especially the active site of the proteolytic antibody, which may reside in the light chain of the antibody. In some embodiments, the active site is located in the CDRs of the light chain. Thus, a wide variety of assays are available and well known in the art for identifying halogen phosphonate monoester probes, immobilizing reagents, and antigen conjugates are capable of binding to proteolytic antibodies.

In an exemplary embodiment, a proteolytic antibody is purified using SDS-PAGE gel chromatography. The antibody may be transferred to a nitrocellulose paper by electroblotting. After blocking the nitrocellulose, the halogen phosphonate monoester probe, immobilizing reagent, or antigen conjugate is contacted with the nitrocellulose paper containing the proteolytic antibody. After washing with the appropriate solvent, a detectable signal is detected indicating the presence of a bound halogen phosphonate monoester probe, immobilizing reagent, or antigen conjugate. Visualization may be accomplished using any appropriate technique as described above (e.g. detection of a labeling moiety or treatment with a proteolytic antibody substrate that produces a detectable signal).

Alternatively, identification of a halogen phosphonate monoester that binds to proteolytic antibodies may be accomplished by simply incubating purified proteolytic antibodies with the halogen phosphonate monoester probe, immobilizing reagent, or antigen conjugate. The reaction mix is then subjected to a separation method, such as high-performance liquid chromatography or capillary electrophoresis, to separate the unbound components from the antibody-phosphonate complexes. Subsequent detection of the complex using an appropriate detection method, such as mass spectrometry or nuclear magnetic resonance, identifies proteolytic antibody binders.

Other exemplary assays are provided in detail in the examples below.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the β-secretase inhibitors of the present invention are equally applicable to the methods of treating disease states and/or the pharmaceutical compositions described herein. All publications, patents, and patent applications cited herein are hereby incorporated by reference in their entirety for all purposes.

### EXAMPLES

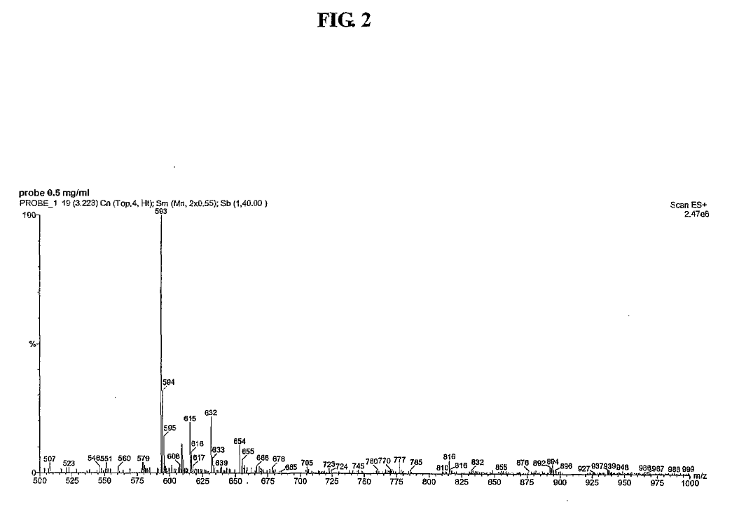

Synthesis of a halogen phosphonate monoester probes, immobilizing reagents, and/or antigen conjugates. Halogen phosphonate monoester probes, immobilizing reagents, and/or antigen conjugates can be synthesized using a method first described by Liu (Proc. Natl. Acad. Sci. 96(26):14694, 1999). As shown in Scheme 1 below, undecylenic acid (1) was esterified with isobutyl alcohol under acid catalysis to yield the isobutyl ester (2) (95% yield, purified by distillation) which was then reduced with metallic sodium by the Boulevenault-Blanc procedure to give 10-Undecen-1-ol (3) (82% yield, purified by distillation). The alcohol was then iodinated by reaction with triphenylphosphine, iodine and imidazole to yield 1-iodo-10 undecene (4) (90% yield, purified by distillation). Reaction of the iodide with triethyl phosphite under reflux afforded the diethoxy phosphonite (5) (87% yield, purified by distillation), which was then converted to the ethoxyhydroxy phosphonite (6) by treatment with trimethylsilyl bromide (TMSBr) (56% yield). The double bond of (6) was oxidatively cleaved with ruthenium trichloride and sodium periodate to yield the terminal carboxylate (7). (81% yield, purified by crystallization of the Na salt). Treatment of (7) with excess Bis-(2-methoxyethyl)aminosulfur trifluoride yielded the difluoro compound (8) (not isolated). The identity of compounds 6 and 7 was confirmed by FTIR, MALDI, and C¹³ NMR. Treatment of (8) with N-hydroxysuccinimide afforded the active ester (9), which is then immediately reacted with 5-(biotinamido) pentylamine (10). (obtained from Pierce Chemical) to generate the fluorophosphonate monoester immobilizing reagent/probe (11). This compound was analyzed by electrospray mass spectrometry and was shown to have the expected formula weight of 593 Da (see FIG. 2).

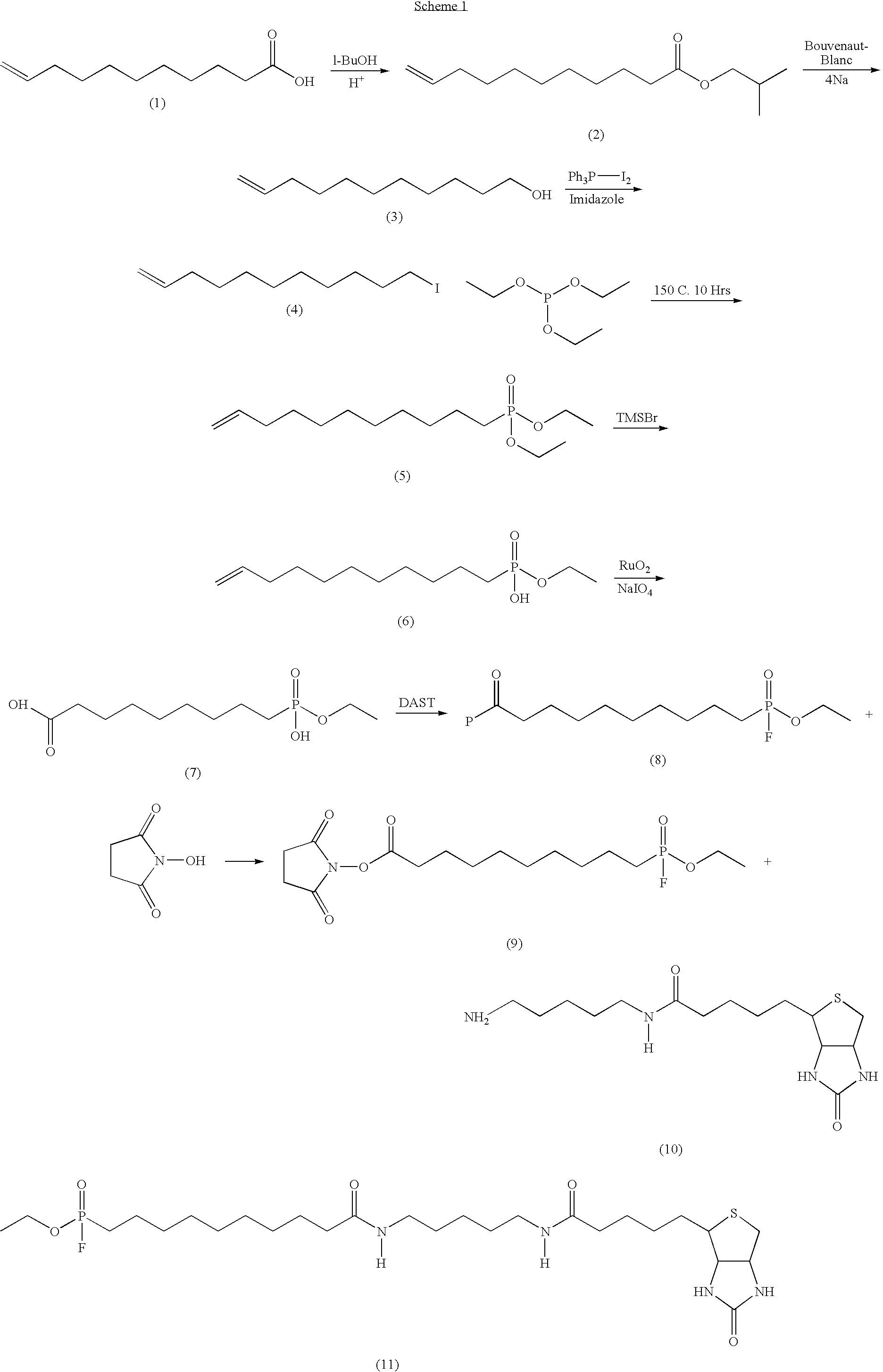

Detection of proteolytic antibody-phosphonate conjugates. The conjugates between halogen phosphonate monoester probes and/or immobilizing reagents and proteolytic antibodies can be detected and analyzed by a number of different methods. For Western blotting analysis, conventional conditions are employed; quenching can be performed with conventional quenching media, e.g. 2×SDS-PAGE loading buffer (reducing), heated for 5-10 min at 80° C. and then run on an SDS-PAGE gel (5-14% acrylamide). After transferring the protein from the gel to a nitrocellulose paper by electroblotting, the blot is: 1) blocked for 15-60 min with 2% bovine serum albumin in TBS-Tween; 2) incubated with avidin-enzyme conjugate (where a biotin immobilizing agent is employed, e.g. horse radish peroxidase) for sufficient time for complex formation (1-2 hrs); 3) washed with TBS-Tween to remove non-specifically bound receptor-enzyme conjugate; 4) treated with an appropriate enzyme substrate for production of a detectable signal; and 5) detecting the site on the blot of the halogen phosphonate immobilizing reagent bound to antibody. One may analyze blots using a chemiluminescence detection system, such as the Lumi-Imager (Roche).

Alternatively, identification of target proteins by avidin affinity purification may be performed. After incubation of protein sample (0.5-2.5 mL, 0.5-1.0 mg/mL) with the halogen phosphonate monoester immobilizing reagents, the sample is diluted to 2.5 mL in Tris or phosphate buffer and passed over a PD-10 size exclusion column to remove excess unreacted halogen phosphonate monoester immobilizing reagent. The protein is collected from the column in 3.5 mL of buffer, treated with SDS (final concentration of 0.5%), and heated for 10 min at 80° C. The sample is then diluted 2.5× and incubated with 100 microliters of avidin agarose beads (Sigma) for 1-4 hours at room temp. The beads are then washed with several volumes of buffer to remove unbound protein. The proteolytic antibody-phosphonate conjugates are eluted with 100 microliters of standard SDS-PAGE loading buffer by heating at 90° C. for 5 minutes. The eluted antibodies are run on an SDS-PAGE gel and proteolytic antibody-phosphonate conjugates identified by staining and/or avidin blotting, excised from the gel, digested with trypsin, and the resulting peptide mixture characterized by MALDI and/or electrospray mass spectrometry. The mass spec information is used in database searches to identify the proteolytic antibody-phosphonate conjugates.

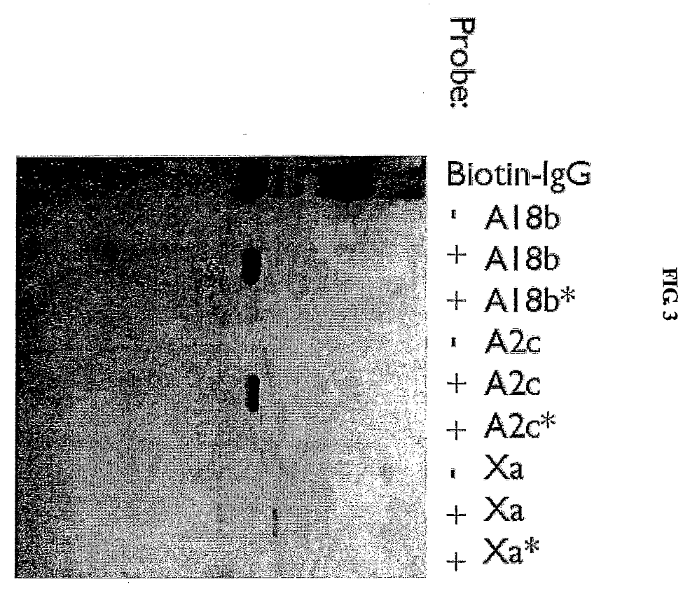

Halogen phosphonate monoester immobilizing reagent binding to an antibody. The kappa light chain A18b proteolytic fragments was cloned and fused to JK1 as described in Smider, 2003 U.S. Pat. App. No. 60/501,073 (incorporated herein by reference in its for all purposes). It was expressed in E. coli periplasm fused to a C-terminal 6-histidine tag. The V_{L} was purified over immobilized nickel affinity columns according to the manufacturers instructions (Invitrogen, Carlsbad, Calif.). Biotinylated fluorophosphonate monoester immobilizing reagent (10 μM) was added to 100 ng antibody light chain (FIG. 3, left panel, lane 2) for 5 minutes at room temperature, then quenched with 2×SDS-PAGE loading buffer and heated to 94° C. for 3 minutes. As controls, the V_{L} was denatured prior to addition of the probe (lane 3), or incubated without probe (lane 1). Similarly, the known serine protease Factor Xa was also incubated with the probe (lane 5), without the probe (lane 4), or heat denatured prior to addition of the probe (lane 6). These mixtures were run on a 15% SDS-PAGE gel, transferred to a nylon membrane, blocked for 45 minutes with 3% bovine serum albumin, and incubated with streptavidin conjugated alkaline phosphatase for 1 hour. The membrane was developed with NBT/BCIP reagent. Identification of covalently binding proteolytic antibody is illustrated in FIG. 3. Lanes 2 and 5 indicate binding of the immobilizing reagent to the V_{L} and Factor Xa, whereas denatured protein (lanes 3 and 6) did not appreciably bind the immobilizing reagent. This indicates that an active site is necessary for the covalent attachment of the immobilizing reagent to the protein. As controls, the same reactions were run on SDS-PAGE and silver stained to show that the protein content in the reactions was identical (FIG. 3, right panel).

What is claimed is:

1. A method for detecting the presence of a proteolytic antibody comprising:
(a) contacting a proteolytic antibody with a halogen phosphonate monoester probe, said halogen phosphonate monoester probe comprising a detectable label;
(b) allowing said halogen phosphonate monoester probe to covalently bind to said proteolytic antibody; and
(c) after step (b), detecting said detectable label thereby detecting the presence of the proteolytic antibody.

2. The method of claim 1, wherein said halogen phosphonate monoester probe has the formula: 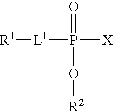
wherein
X is a halogen;
L¹ is selected from the group consisting of a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene, wherein L¹ is not attached to the phosphorus through an oxygen heteroatom;
R¹ is a detectable label; and
R² is selected from the group consisting of a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

3. The method of claim 2, wherein said detectable label is selected from the group consisting of a mass tag label, radioisotopic label, metal chelate label, luminescent label, electroactive label, enzyme modulator label, photosensitizer label, or electron transfer label.

4. The method of claim 2, wherein
L¹ is selected from the group consisting of a bond, substituted or unsubstituted C₁-C₅₀ alkylene, substituted or unsubstituted 2 to 50 membered heteroalkylene, substituted or unsubstituted C₃-C₈ cycloalkylene, substituted or unsubstituted 3 to 8 membered heterocycloalkylene, substituted or unsubstituted arylene, and substituted or unsubstituted heteroarylene;
R² is selected from the group consisting of hydrogen, substituted or unsubstituted C₁-C₂₀ alkyl, substituted or unsubstituted 2 to 20 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

5. The method of claim 2, wherein
L¹ is selected from the group consisting of a bond, substituted or unsubstituted C₁-C₄₀ alkylene, and substituted or unsubstituted 2 to 40 membered heteroalkylene;
R² selected from the group consisting of hydrogen, substituted or unsubstituted C₁-C₁₀ alkyl, substituted or unsubstituted 2 to 10 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

6. The method of claim 2, wherein
L¹ is selected from the group consisting of a bond; unsubstituted C₁-C₄₀ alkylene; unsubstituted 2 to 40 membered heteroalkylene; and
C₁-C₄₀ alkylene or 2 to 40 membered heteroalkylene substituted with a substituent,
said substituent is independently selected from the group consisting of an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

7. The method of claim 2, wherein
R² is selected from the group consisting of hydrogen; unsubstituted C₁-C₁₀ alkyl; unsubstituted 2 to 10 membered heteroalkyl; unsubstituted C₃-C₈ cycloalkyl; unsubstituted 3 to 8 membered heterocycloalkyl; unsubstituted aryl; unsubstituted heteroaryl; and
C₁-C₁₀ alkyl, 2 to 10 membered heteroalkyl, C₃-C₈ cycloalkyl, 3 to 8 membered heterocycloalkyl, aryl, or heteroaryl substituted with a substituent,
said substituent independently selected from oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, and unsubstituted heteroaryl.

8. The method of claim 2, wherein
L¹ is a 2 to 40 membered heteroalkylene substituted with an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl; and
R² is unsubstituted C₁-C₁₀ alkyl.

9. The method of claim 1, wherein said proteolytic antibody is present in a sample comprising a plurality of antibodies.

10. The method of claim 1, wherein said proteolytic antibody forms part of an antibody library or synthetic antibody library.

11. A method for immobilizing a proteolytic antibody comprising:
(a) contacting a proteolytic antibody with a halogen phosphonate monoester immobilizing reagent, said halogen phosphonate monoester immobilizing reagent comprising a solid support or immobilizing moiety;
(b) allowing the immobilizing moiety to bind to a complimentary solid support;
(c) allowing said halogen phosphonate monoester immobilizing reagent to covalently bind to said proteolytic antibody, thereby immobilizing said proteolytic antibody.

12. The method of claim 11, wherein said halogen phosphonate monoester immobilizing reagent has the formula: 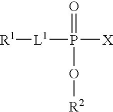
wherein
L¹ is selected from the group consisting of a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene, wherein L¹ is not attached to the phosphorus through an oxygen heteroatom;
R¹ is an immobilizing moiety or solid support;
X is a halogen; and
R² is selected from the group consisting of a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

13. The method of claim 12, wherein said immobilizing moiety is selected from the group consisting of an affinity tag or a crosslinking group.

14. The method of claim 13, wherein said affinity tag is selected from the group consisting of biotin, deiminobiotin, dethiobiotin, vicinal diol, digoxigenin, maltose, oligohistidine, glutathione, 2,4-dinitrobenzene, phenylarsenate, ssDNA, dsDNA, polyhistidine, and a hapten.

15. The method of claim 12, wherein
L¹ is selected from the group consisting of a bond, substituted or unsubstituted C₁-C₅₀ alkylene, substituted or unsubstituted 2 to 50 membered heteroalkylene, substituted or unsubstituted C₃-C₈ cycloalkylene, substituted or unsubstituted 3 to 8 membered heterocycloalkylene, substituted or unsubstituted arylene, and substituted or unsubstituted heteroarylene;
R² is selected from the group consisting of hydrogen, substituted or unsubstituted C₁-C₂₀ alkyl, substituted or unsubstituted 2 to 20 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

16. The method of claim 12, wherein
L¹ is selected from the group consisting of a bond, substituted or unsubstituted C₁-C₄₀ alkylene, and substituted or unsubstituted 2 to 40 membered heteroalkylene;
R² selected from the group consisting of hydrogen, substituted or unsubstituted C₁-C₁₀ alkyl, substituted or unsubstituted 2 to 10 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

17. The method of claim 12, wherein
L¹ is selected from the group consisting of a bond; unsubstituted C₁-C₄₀ alkylene; unsubstituted 2 to 40 membered heteroalkylene; and
C₁-C₄₀ alkylene or 2 to 40 membered heteroalkylene substituted with a substituent,
said substituent is independently selected from the group consisting of an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

18. The method of claim 12, wherein
R² is selected from the group consisting of hydrogen; unsubstituted C₁-C₁₀ alkyl; unsubstituted 2 to 10 membered heteroalkyl; unsubstituted C₃-C₈ cycloalkyl; unsubstituted 3 to 8 membered heterocycloalkyl; unsubstituted aryl; unsubstituted heteroaryl; and
C₁-C₁₀ alkyl, 2 to 10 membered heteroalkyl, C₃-C₈ cycloalkyl, 3 to 8 membered heterocycloalkyl, aryl, or heteroaryl substituted with a substituent,
said substituent independently selected from oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, and unsubstituted heteroaryl.

19. The method of claim 12, wherein
L¹ is a 2 to 40 membered heteroalkylene substituted with an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl; and
R² is unsubstituted C₁-C₁₀ alkyl.

20. A method for producing a proteolytic antibody in a subject comprising:
(a) administering a halogen phosphonate monoester antigen conjugate to said subject;
(b) allowing said subject to produce proteolytic antibodies to said halogen phosphonate monoester antigen conjugate thereby producing said proteolytic antibody in said subject.

21. The method of claim 20, further comprising isolating said proteolytic antibodies from said subject.

22. The method of claim 20, wherein said halogen phosphonate monoester antigen conjugate has the formula: 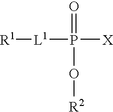
wherein
X is a halogen;
L¹ is selected from the group consisting of a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene, wherein L¹ is not attached to the phosphorus through an oxygen heteroatom;
R¹ is an antigen moiety;
R² is selected from the group consisting of a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

23. The method of claim 22, wherein said antigen moiety is a peptide antigen moiety.

24. The method of claim 22, wherein said antigen moiety is a non-hydrolytic peptide antigen moiety.

25. The method of claim 22, wherein said antigen moiety is selected from the group consisting of a growth factor, cell surface receptor, cytokine, and immunoglobulin.

26. The method of claim 22, wherein said antigen moiety is selected from the group consisting of TNFα, vascular endothelial growth factor, interferon-γ, and CD20.

27. The method of claim 22, wherein
L¹ is selected from the group consisting of a bond, substituted or unsubstituted C₁-C₅₀ alkylene, substituted or unsubstituted 2 to 50 membered heteroalkylene, substituted or unsubstituted C₃-C₈ cycloalkylene, substituted or unsubstituted 3 to 8 membered heterocycloalkylene, substituted or unsubstituted arylene, and substituted or unsubstituted heteroarylene;
R² is selected from the group consisting of hydrogen, substituted or unsubstituted C₁-C₂₀ alkyl, substituted or unsubstituted 2 to 20 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

28. The method of claim 22, wherein
L¹ is selected from the group consisting of a bond, substituted or unsubstituted C₁-C₄₀ alkylene, and substituted or unsubstituted 2 to 40 membered heteroalkylene;
R² selected from the group consisting of hydrogen, substituted or unsubstituted C₁-C₁₀ alkyl, substituted or unsubstituted 2 to 10 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

29. The method of claim 22, wherein
L¹ is selected from the group consisting of a bond; unsubstituted C₁-C₄₀ alkylene; unsubstituted 2 to 40 membered heteroalkylene; and
C₁-C₄₀ alkylene or 2 to 40 membered heteroalkylene substituted with a substituent,
said substituent is independently selected from the group consisting of an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

30. The method of claim 22, wherein
R² is selected from the group consisting of hydrogen; unsubstituted C₁-C₁₀ alkyl; unsubstituted 2 to 10 membered heteroalkyl; unsubstituted C₃-C₈ cycloalkyl; unsubstituted 3 to 8 membered heterocycloalkyl; unsubstituted aryl; unsubstituted heteroaryl; and
C₁-C₁₀ alkyl, 2 to 10 membered heteroalkyl, C₃-C₈ cycloalkyl, 3 to 8 membered heterocycloalkyl, aryl, or heteroaryl substituted with a substituent,
said substituent independently selected from oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, and unsubstituted heteroaryl.

31. The method of claim 22, wherein
L¹ is a 2 to 40 membered heteroalkylene substituted with an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl; and
R² is unsubstituted C₁-C₁₀ alkyl.

32. A halogen phosphonate monoester antigen conjugate having the formula: 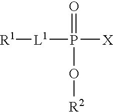
wherein
X is a halogen;
L¹ is selected from the group consisting of a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene, wherein L¹ is not attached to the phosphorus through an oxygen heteroatom;
R¹ is an antigen moiety; and
R² is selected from the group consisting of a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

33. The halogen phosphonate monoester antigen conjugate of claim 32, wherein
L¹ is selected from the group consisting of a bond, substituted or unsubstituted C₁-C₅₀ alkylene, substituted or unsubstituted 2 to 50 membered heteroalkylene, substituted or unsubstituted C₃-C₈ cycloalkylene, substituted or unsubstituted 3 to 8 membered heterocycloalkylene, substituted or unsubstituted arylene, and substituted or unsubstituted heteroarylene;
R² is selected from the group consisting of hydrogen, substituted or unsubstituted C₁-C₂₀ alkyl, substituted or unsubstituted 2 to 20 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

34. The halogen phosphonate monoester antigen conjugate of claim 32, wherein
L¹ is selected from the group consisting of a bond, substituted or unsubstituted C₁-C₄₀ alkylene, and substituted or unsubstituted 2 to 40 membered heteroalkylene;
R² selected from the group consisting of hydrogen, substituted or unsubstituted C₁-C₁₀ alkyl, substituted or unsubstituted 2 to 10 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

35. The halogen phosphonate monoester antigen conjugate of claim 32, wherein
L¹ is selected from the group consisting of a bond; unsubstituted C₁-C₄₀ alkylene; unsubstituted 2 to 40 membered heteroalkylene; and
C₁-C₄₀ alkylene or 2 to 40 membered heteroalkylene substituted with a substituent,
said substituent is independently selected from the group consisting of an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

36. The halogen phosphonate monoester antigen conjugate of claim 32, wherein
R² is selected from the group consisting of hydrogen; unsubstituted C₁-C₁₀ alkyl; unsubstituted 2 to 10 membered heteroalkyl; unsubstituted C₃-C₈ cycloalkyl; unsubstituted 3 to 8 membered heterocycloalkyl; unsubstituted aryl; unsubstituted heteroaryl; and
C₁-C₁₀ alkyl, 2 to 10 membered heteroalkyl, C₃-C₈ cycloalkyl, 3 to 8 membered heterocycloalkyl, aryl, or heteroaryl substituted with a substituent,
said substituent independently selected from oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, and unsubstituted heteroaryl.

37. The halogen phosphonate monoester antigen conjugate of claim 32, wherein
L¹ is a 2 to 40 membered heteroalkylene substituted with an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl; and
R² is unsubstituted C₁-C₁₀ alkyl.

38. A proteolytic antibody immobilization system comprising:
(a) a halogen phosphonate monoester immobilizing reagent; and
(b) a solid support.

39. The proteolytic antibody immobilization system of claim 38, wherein said halogen phosphonate monoester immobilizing reagent has the formula: 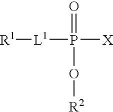
wherein
L¹ is s selected from the group consisting of a bond, substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene, wherein L¹ is not attached to the phosphorus through an oxygen heteroatom;
R¹ is an immobilizing moiety;
X is a halogen; and
R² is selected from the group consisting of a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

40. The proteolytic antibody immobilization system of claim 38, wherein said immobilizing moiety is selected from the group consisting of an affinity tag or a crosslinking group.

41. The proteolytic antibody immobilization system of claim 40, wherein
if said immobilizing moiety is an affinity tag, then said solid support comprises an affinity tag binder; and
if said immobilizing moiety is a crosslinking group, then said solid support comprises a crosslinking group.

42. The proteolytic antibody immobilization system of claim 39, wherein said immobilizing moiety is selected from the group consisting of an affinity tag or a crosslinking group.

43. The proteolytic antibody immobilization system of claim 39, wherein said affinity tag is selected from the group consisting of biotin, deiminobiotin, dethiobiotin, vicinal diol, digoxigenin, maltose, oligohistidine, glutathione, 2,4-dinitrobenzene, phenylarsenate, ssDNA, dsDNA, polyhistidine, and a hapten.

44. The proteolytic antibody immobilization system of claim 39, wherein
L¹ is selected from the group consisting of a bond, substituted or unsubstituted C₁-C₅₀ alkylene, substituted or unsubstituted 2 to 50 membered heteroalkylene, substituted or unsubstituted C₃-C₈ cycloalkylene, substituted or unsubstituted 3 to 8 membered heterocycloalkylene, substituted or unsubstituted arylene, and substituted or unsubstituted heteroarylene;
R² is selected from the group consisting of hydrogen, substituted or unsubstituted C₁-C₂₀ alkyl, substituted or unsubstituted 2 to 20 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

45. The proteolytic antibody immobilization system of claim 39, wherein
L¹ is selected from the group consisting of a bond, substituted or unsubstituted C₁-C₄₀ alkylene, and substituted or unsubstituted 2 to 40 membered heteroalkylene;
R² selected from the group consisting of hydrogen, substituted or unsubstituted C₁-C₁₀ alkyl, substituted or unsubstituted 2 to 10 membered heteroalkyl, substituted or unsubstituted C₃-C₈ cycloalkyl, substituted or unsubstituted 3 to 8 membered heterocycloalkyl, substituted or unsubstituted aryl, and substituted or unsubstituted heteroaryl.

46. The proteolytic antibody immobilization system of claim 39, wherein
L¹ is selected from the group consisting of a bond; unsubstituted C₁-C₄₀ alkylene; unsubstituted 2 to 40 membered heteroalkylene; and
C₁-C₄₀ alkylene or 2 to 40 membered heteroalkylene substituted with a substituent,
said substituent is independently selected from the group consisting of an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

47. The proteolytic antibody immobilization system of claim 39, wherein
R² is selected from the group consisting of hydrogen; unsubstituted C₁-C₁₀ alkyl; unsubstituted 2 to 10 membered heteroalkyl; unsubstituted C₃-C₈ cycloalkyl; unsubstituted 3 to 8 membered heterocycloalkyl; unsubstituted aryl; unsubstituted heteroaryl; and
C₁-C₁₀ alkyl, 2 to 10 membered heteroalkyl, C₃-C₈ cycloalkyl, 3 to 8 membered heterocycloalkyl, aryl, or heteroaryl substituted with a substituent,
said substituent independently selected from oxy, unsubstituted C₁-C₂₀ allyl, unsubstituted 2 to 20 membered heteroalkyl, unsubstituted C₃-C₈ cycloalkyl, unsubstituted 3 to 8 membered heterocycloalkyl, unsubstituted aryl, and unsubstituted heteroaryl.

48. The proteolytic antibody immobilization system of claim 39, wherein
L¹ is a 2 to 40 membered heteroalkylene substituted with an oxy, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl; and
R² is unsubstituted C₁-C₁₀ alkyl.
